(19) United States
(12) Patent Application Publication
(10) Pub. No.: US 2009/0099469 A1
(43) Pub. Date: Apr. 16, 2009

(54) Wireless ECG/EKG patient telemetry monitoring system

(76) Inventor: Pamela A. Flores, Westlake, LA (US)

Correspondence Address: Raymond M. Galasso;Galasso & Associates, LP, P.O.Box 26503, Austin, TX, 78755-0503, US

(21) Appl. No.: 11/974,131

(22) Filed: Oct. 11, 2007

(51) Int. Cl. A61B 5/0408

(52) U.S. Cl. 600/523

(57) ABSTRACT

An ECG/EKG patient telemetry monitoring system is disclosed, the system configured for monitoring the cardiac activity of a patient. The wireless ECG patient telemetry monitoring system includes a plurality of self-contained wireless electrodes adapted to adhesively secure to the skin of the torso of the patient and to measure electric potential on the skin as related to electrical activity of the heart. ECG signals from the electrodes are wirelessly communicated to a central monitor, which provides graphical ECG voltage potential versus time plots for a technician, nurse, or physician.

### FIELD OF THE DISCLOSURE

The disclosures made herein relate generally to ECG/EKG monitoring equipment for monitoring and evaluating cardiac activity of a patient and, more particularly, to a wearable ECG/EKG patient telemetry monitoring system which permits the monitored patient to freely roam or move about without the encumbrances of wired electrodes.

### BACKGROUND

Clinical care and medical health evaluations often involve the use of electrocardiograms (ECG). An ECG is a highly useful tool in the evaluation of a patient's cardiac activity, including heart rate and rhythms. The ECG is indicative of the conduction of low level electrical impulses across the myocardium, the low level impulses sometimes referred to as cardiac impulse. An ECG typically receives signals from a plurality of sensors, typically three, five or twelve sensors, that are temporarily secured and positioned on the skin of the patient, typically on the torso/chest of the patient. The ECG measures low level voltage signals appearing between pairs of electrodes and processes these signals to provide a two dimensional graphical display of the detected signal voltage verses time to provide a graphical output indicative of cardiac activity. Such ECG techniques, sometimes called surface ECG, rely upon the detection of low voltage signals at the surface of the skin of the patient, in contrast to internal ECG which places electrodes directly on the cardiac tissue and therefore requires invasive procedures.

The ECG is a method of recording the electrical activity of the heart. Each heartbeat is caused by a section of the heart generating an electrical signal, which then conducts through special pathways to all parts of the heart. These electrical signals, in an attenuated form, can be detected at the skin of the patient and recorded. The complex nature of each heartbeat begins in a specialized area of the right atrium called the sinus node. The sinus node is one of the major elements in the cardiac conduction system, the system that controls the heart rate. The autonomic nervous system, the same part of the nervous system that controls blood pressure, controls the firing of the sinus node to trigger the start of the cardiac cycle. The sinus node starts each heartbeat by generating a small amount of electricity via simple electrochemical potentials that are induced by salt concentrations. This signaling spreads into the muscle cells of the atria. This causes these upper chambers to contract. Next, the electrical activity moves into the junctions between the atria and ventricles, the heart's main pumping chambers. This area is called the atria ventricular node or A-V node. The A-V node acts as a relay station. It takes the signal coming from the atria, delays it slightly, then passes it into the ventricles, which causes the ventricles to contract or beat. When the ventricles beat, blood is pumped throughout the body. This process repeats at periodic intervals.

The EKG/ECG can assist physicians in diagnosing abnormalities of the heart such as abnormal heart rates or rhythms, abnormal size of parts of the heart such as enlargement or atrophy, abnormal conduction pathways, and location of damaged muscle.

A limitation of conventional EKG/ECG equipment using wired electrodes is that the patient is hard wired to the monitor, thereby limiting the mobility of the patient.

Another limitation of conventional EKG/ECG equipment is false detection of cardiac alarms due to the lead wires rubbing together as well as poor transmission signal in lead wires.

Another limitation of conventional EKG/ECG equipment is the tangling of lead wires and the time spent attaching and detaching lead wires when moving a patient in the care facility.

A very serious limitation of conventional EKG/ECG equipment is exposed by a study conducted at the University of Wisconsin which reported that 77% of ECG telemetry leads that were cleaned by standard hospital methods were found to be contaminated with one or more antibiotic-resistant pathogens. These pathogens enter the blood stream through open wounds, placing at risk the health of injured and post-surgical patients.

Therefore, a wireless ECG patient telemetry monitoring system that eliminates the use of wired EKG/ECG electrodes on patients, that permits mobility of the patient, and that utilizes disposable electrodes that communicate wirelessly with the EKG/ECG monitoring equipment, such a ECG patient telemetry monitoring system would be novel and useful.

### SUMMARY OF THE DISCLOSURE

Accordingly, embodiments of the inventive disclosures made herein comprise various embodiments of a wireless ECG patient telemetry monitoring system for monitoring the cardiac activity of a patient.

In one or more embodiments of the inventive disclosures made herein, the wireless ECG patient telemetry monitoring system includes a plurality of self-contained wireless electrodes adapted to adhesively secure to the skin of the torso of the patient and to measure electric potential on the skin as related to electrical activity of the heart.

Each wireless electrode includes a capsular housing which encloses a power supply, such as a battery; an ECG sensor secured proximate to the skin contacting portion of the sensor; a computer processor and custom embedded software configured and adapted to execute upon the processor; and a wireless signal data transmitter configured to transmit the encoded ECG sensors signals to an ECG telemetry data receiver. The software and processor are adapted and configured to process the ECG signals received from the ECG sensor, formatting the data for wireless transmission to an ECG telemetry data receiver.

An adhesive patch is secured to the skin-contacting portion of the sensor. The adhesive patch is adapted to removeably and adhesively secure the wireless electrode to the skin of the patient.

At least one telemetry receiver is provided, configured and adapted to receive the ECG sensor signals from the plurality of wireless electrodes as transmitted by the transmitters of the wireless electrodes. At least one central ECG monitor is provided and configured to receive the ECG signals from the telemetry receiver. The ECG monitor includes a human readable display device for graphically displaying the ECG signals as indicative of cardiac activity of the patient. Further included is a computer processor and software adapted to execute upon the processor, wherein the computer processor and software is configured and adapted to process the ECG signals and to graphically display the ECG signals on the display device in a format that is comprehendible by a technician, nurse or physician.

It is an object of the present invention to provide a wireless ECG patient telemetry monitoring system that eliminates the use of wired electrodes on a patient.

It is another object of the present invention to provide a wireless ECG patient telemetry monitoring system that permits unencumbered mobility of the patient while undergoing ECG monitoring.

It is another object of the present invention to provide a wireless ECG patient telemetry monitoring system that utilizes disposable electrodes that communicate wirelessly with the monitoring equipment.

It is another object of the present invention to provide a wireless ECG patient telemetry monitoring system that utilizes disposable wireless electrodes, thereby eliminating the problem of infection of the patient from conventional multi-use electrodes contaminated with antibiotic-resistant pathogens.

These and other objects of the invention made herein will become readily apparent upon further review of the following specification and associated drawings.

### BRIEF DESCRIPTION OF THE DRAWINGS

The drawings show a form of the invention that is presently preferred; however, the invention is not limited to the precise arrangement shown in the drawings.

FIG. 1 depicts a set of five self-contained wireless electrodes with labels or placement designators for removeably attaching to the chest or torso of a patient, in accordance with the inventive disclosures herein.

FIG. 2 depicts a patient with five self-contained wireless electrodes removeably secured to the chest or torso of a patient and wirelessly in communication with a central monitor, in accordance with the inventive disclosures herein.

FIG. 3 depicts a side view of a self-contained wireless electrode, in accordance with the inventive disclosures herein.

FIG. 4 is a high-level schematic block diagram of one exemplary wireless ECG patient telemetry monitoring system, in accordance with the inventive disclosures herein.

### DETAILED DESCRIPTION OF THE DRAWINGS

In preparation for explaining the details of the present inventive disclosure, it is to be understood by the reader that the invention is not limited to the presented details of the construction, materials and embodiments as illustrated in the accompanying drawings, as the invention concepts are clearly capable of other embodiments and of being practiced and realized in various ways by applying the disclosure presented herein.

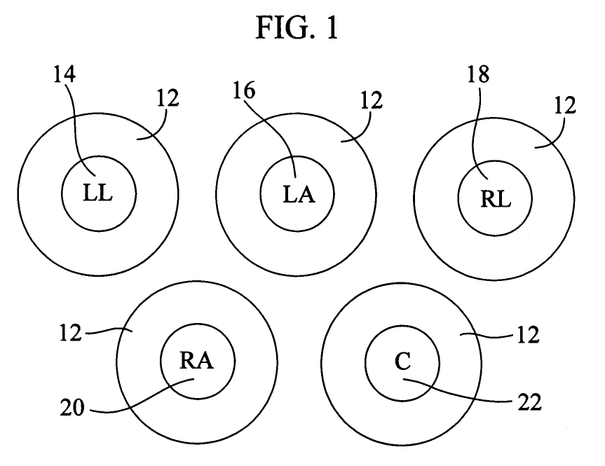

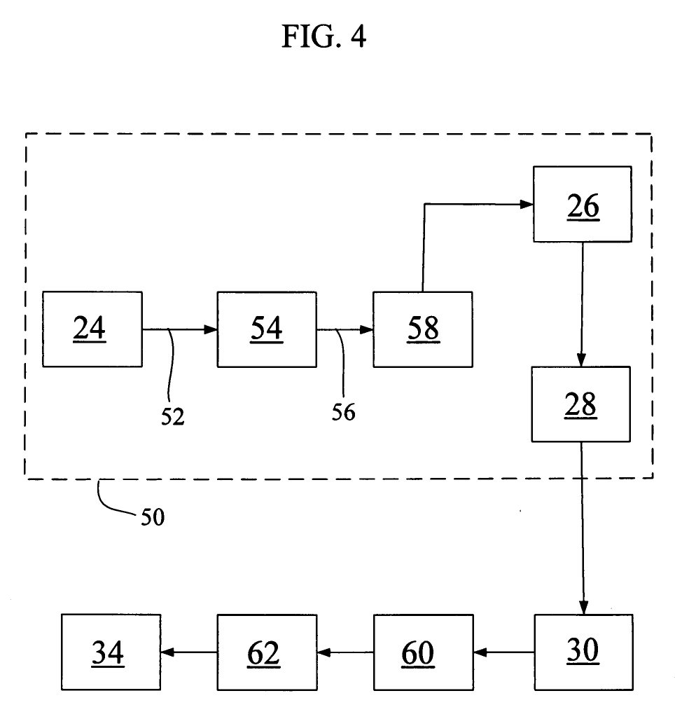

FIG. 1 depicts a set of five self-contained wireless electrodes with labels or placement designators for removeably attaching to the chest or torso of a patient, in accordance with the inventive disclosures herein. Each electrode consists of a capsular housing (14, 16, 18, 20 or 22) secured to an adhesive patch 12. The capsular housing includes an ECG sensor 24 (see FIG. 4), such as a direct contact voltage sensor or a capacitive sensor. Also included is a power supply such as a battery, a computer processor with embedded software 26 (see FIG. 4) adapted and configured to process ECG signals from the ECG sensor 24 for wireless transmission, and a wireless signal data transmitter 28 (see FIG. 4) configured to transmit the encoded ECG sensors signals to an ECG telemetry data receiver 30 (see FIG. 4). The adhesive patches are adapted to removeably and adhesively secure the wireless electrode to the skin of the patient.

In some embodiments, each of the five wireless electrodes are uniquely identified by labels, the labels including LL, LA, RL, RA, and C. The labeled electrodes are referred to as placement designators. In certain embodiments each labeled electrode or placement designator is provided in color that uniquely identifies the placement designator. In at least one embodiment the following placement designator color identification scheme is used: The LL placement designator is red in color. The LA placement designator is gray in color. The RL placement designator is green in color. The RA placement designator is white in color. The C placement designator is brown in color.

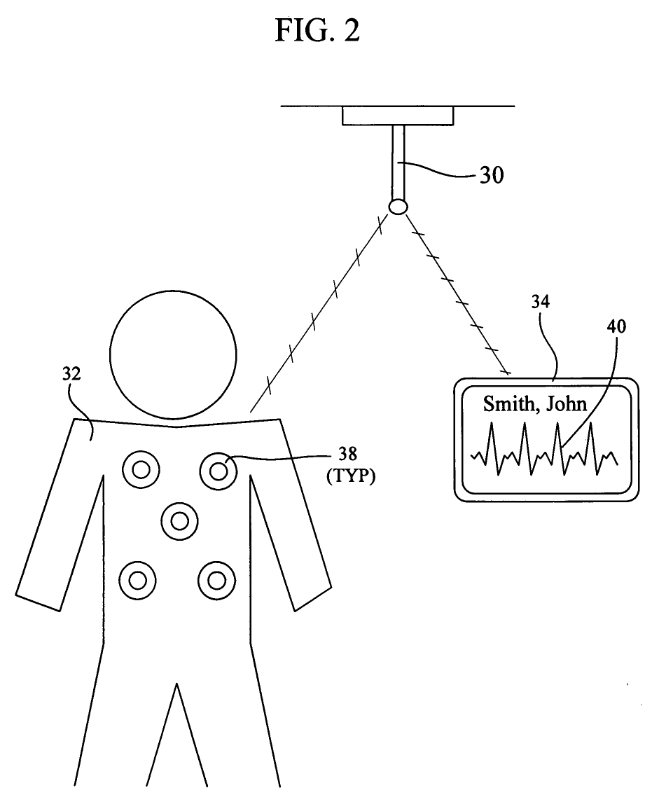

FIG. 2 depicts a patient with five self-contained wireless electrodes 38, in particular the wireless electrodes of FIG. 1, removeably secured to the chest or torso of a patient 32, in accordance with the inventive disclosures herein. Each wireless electrode 38 communicates ECG data wirelessly with a telemetry receiver 30. The ECG data received at the receiver 30 is processed and correlated with other ECG data from other sensors 38 on the patient 32 by the central monitor 34 for graphics display of an ECG plot 40 on the monitor 34.

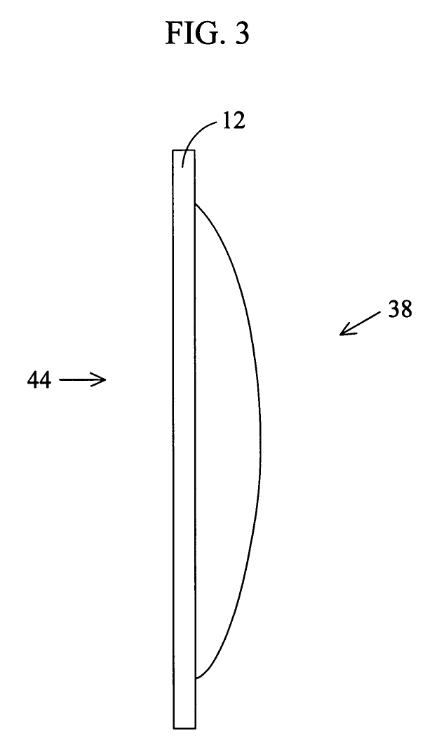

FIG. 3 depicts a side view of a self-contained wireless electrode 38 (such as 14, 16, 18, 20 or 22), in accordance with the inventive disclosures herein. The wireless electrode or placement designator 38 has a skin-contacting portion 44 configured for adhesively attaching, the wireless electrode 38 to the patient 32 (see FIG. 2) by the adhesive patch 12.

FIG. 4 is a high-level schematic block diagram of one non-limiting particular example of a wireless ECG patient telemetry monitoring system, in accordance with the inventive disclosures herein. In the exemplary embodiment, the ECG sensor 24 provides a low level ECG signal 52 to an amplifier 54. The amplifier 56 provides an amplified ECG signal to the voltage to frequency converter 58. The voltage to frequency converter 58 may be provides as a separate function as shown, or may be incorporated into processor/software 26. Processor 26 forwards the frequency encoded ECG sensor signals to the transmitter 28. The dashed lines 50 identify components and functions performed in the self-contained wireless electrodes 38. The central monitor includes a receiver 30 receiving ECG sensor signals from transmitter 28. A frequency to voltage converter 60 decodes the received ECG signals into voltage signals, which are processed by processor/software 62 to drive and generate a graphical ECG display on the display device 34. The high-level schematic block diagram of FIG. 4 is intended to schematically illustrate one possible embodiment and is not intended to limit the scope of the invention to the exemplary embodiment presented.

The discussed construction, illustrations and sequence of operation is for one embodiment of the invention, but is in no way limiting to other embodiments. The operating modes may be changed and enhanced without deviating from the intention of this inventive disclosure.

In the preceding detailed description, reference has been made to the accompanying drawings that form a part hereof, and in which are shown by way of illustration specific embodiments in which the invention may be practiced. These embodiments and certain variants thereof have been described in sufficient detail to enable those skilled in the art to practice the invention. It is to be understood that other suitable embodiments may be utilized and that logical, material, and mechanical changes may be made without departing from the spirit or scope of the invention. To avoid unnecessary detail, the description omits certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A wireless ECG patient telemetry monitoring system comprising:
a plurality of self-contained wireless electrodes adapted to adhesively secure to skin of the torso of said patient and measure electric potential on said skin, said wireless electrodes comprising:
a capsular housing comprising:
a power supply, such as a battery;
an ECG sensor secured proximate to a skin contacting portion of said sensor;
a computer processor and embedded software adapted to execute upon said processor, said processor and said software adapted and configured to process ECG signals from said ECG sensor for wireless transmission; and
a wireless signal data transmitter configured to transmit said encoded ECG sensor signals to an ECG telemetry data receiver;
an adhesive patch secured to said skin contacting portion of said sensor, said adhesive patch adapted to removeably and adhesively secure said wireless electrode to said skin of said patient;
at least one telemetry receiver configured and adapted to receive said ECG sensor signals from said wireless electrodes;
at least one central ECG monitor configured to receive said ECG signals from said telemetry receiver, said ECG monitor comprising:
a display device for graphically displaying said ECG signals indicative of cardiac activity of said patient; and
a computer processor and software adapted to execute upon said processor, said computer processor and software configured and adapted to process said ECG signals and graphical display of said ECG signals on said display device;
wherein said wireless electrodes are wearable by said patient; and
wherein said ECG patient telemetry monitoring system is adapted to permit said patient to freely roam without encumbrance of wires.

2. The wireless ECG patient telemetry monitoring system according to claim 1, wherein said plurality of self-contained wireless electrodes is five wireless electrodes.

3. The wireless ECG patient telemetry monitoring system according to claim 2, wherein said five wireless electrodes are uniquely and identifiably labeled: LL, LA, RL, RA, and C; wherein said labeled electrodes are referred to as placement designators; wherein said labels identify positions for attachments of said electrodes to said torso of said patient.

4. The wireless ECG patient telemetry monitoring system according to claim 3, wherein
said LL placement designator is red in color;
said LA placement designator is gray in color;
said RL placement designator is green in color;
said RA placement designator is white in color; and
said C placement designator is brown in color.

5. The wireless ECG patient telemetry monitoring system according to claim 4, wherein said placement designators are disposable.

6. The wireless ECG patient telemetry monitoring system according to claim 5, wherein said placement designators are circular in shape and have a diameter of two centimeters.

7. The wireless ECG patient telemetry monitoring system according to claim 5, wherein said placement designators are square in shape and have a dimension between opposing sides of said square of two centimeters.

8. The wireless ECG patient telemetry monitoring system according to claim 1, wherein said central monitor is adapted to selectively provide a permanent record of cardiac activity of said patient.

9. The wireless ECG patient telemetry monitoring system according to claim 1, wherein said system is designed for use in a residence of said patient.
